(19) United States
(12) Patent Application Publication
(10) Pub. No.: US 2002/0160043 A1
(43) Pub. Date: Oct. 31, 2002

(54) Compositions and method of manufacture for oral dissolvable dosage forms

(76) Inventor: Dennis Coleman, Salt Lake City, UT (US)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 09/798,027

(22) Filed: Feb. 27, 2001

(51) Int. Cl. A61K 9/20; A61K 9/14

(52) U.S. Cl. 424/465; 424/488

(57) ABSTRACT

Compositions and methods of manufacture for dissolvable and nondissolvable drug-containing dosage-forms for noninvasive administration of medicaments through mucosal tissues of the mouth, pharynx, and esophagus of a patient. The dosage-forms are particularly useful in the transmucosal delivery of central nervous system affecting drugs in a dose-to-effect manner such that a sufficient dose is administered to produce a desired effect. A dissolvable drug-containing dosage-form includes a binding agent that is formed into a solid matrix dissolvable in the mouth of the patient, and a pharmacologically effective dose of a central nervous system affecting drug dispersed throughout the matrix. A nondissolvable drug-containing dosage-form includes a drug containment matrix that is nondissolvable in the mouth of the patient, and a central nervous system affecting drug incorporated into the nondissolvable matrix. The dissolvable and nondissolvable drug-containing dosage-forms may include permeation enhancers capable of modifying the permeability of the mucosal tissues of the mouth, pharynx, and esophagus in order to facilitate transmucosal absorption of the drug.

### BACKGROUND OF THE INVENTION

1. The Field of the Invention

The present invention relates to drug-containing lozenges for use in the transmucosal delivery of medicaments to a patient. More particularly, the present invention relates to oral dissolvable drug-containing lozenges, for noninvasive administration of medicaments through the mucosal tissues of the mouth, pharynx, or esophagus of a patient.

2. The Relevant Technology

Numerous advancements have taken place in the field of pharmacology and pharmaceutics with respect to the administration of drugs to treat various conditions. Despite the tremendous advancements in the field, drugs continue to be administered using substantially the same techniques that have been used for many decades. The vast majority of pharmaceutical agents continue to be administered either orally or by injection. Nevertheless, it is frequently found that neither of these administration routes are effective in all cases, and both administration routes suffer from several disadvantages.

Oral administration is probably the most prevalent method of administering pharmacological medicaments. The medicament is generally incorporated into a tablet, capsule, or a liquid base, and then swallowed. The oral administration modality is often preferred because of its convenience. In addition, oral administration is generally nonthreatening, painless, and simple to accomplish for most patients.

Nevertheless, oral administration of drugs suffers from several disadvantages. One disadvantage is that pediatric and geriatric patients frequently have difficulty swallowing pills and other solid dosage-forms, and such patients often refuse to cooperate in swallowing a liquid medication. In addition, for many medicaments, the act of swallowing the medicament often requires fluids, resulting in increased gastric volume and the likelihood of nausea and vomiting.

A further problem with oral administration is that the rate of absorption of the drug into the bloodstream after swallowing varies from patient to patient. The absorption of the drug is dependent upon the movement of the drug from the stomach to the small and large intestines, on the effects of secretions from these organs, and on the resulting pH within the stomach and intestines. Anxiety and stress can dramatically reduce these movements and secretions, prevent or reduce the final effects of the drug, and delay onset of the drug effect.

Most significant is the fact that there is normally a substantial delay between the time of oral administration and the time that the therapeutic effect of the drug begins. As mentioned above, the drug must pass through the gastrointestinal system in order to enter the bloodstream; this typically takes forty-five minutes or longer. Anxiety and stress often increase this delay.

For many applications, such as premedication before surgery, immediate relief from pain or a serious medical condition, or where immediate effectiveness of the drug is required, this delay is unacceptable. In modem outpatient units and operating rooms where rapid turnover of patients is essential for cost containment, extensive delays in the action of a drug may increase costs.

An additional disadvantage of oral administration is that many drugs almost immediately experience metabolism or inactivation. The veins from the stomach and the small and large intestines pass directly through the liver. Thus, drugs entering the bloodstream must first pass through the liver before distribution into the general blood circulation. More than sixty percent of most drugs (and essentially one hundred percent of certain drugs) are removed from the patient's blood stream during this “first pass” through the liver. The result is that oral administration is impractical for many drugs, particularly many central nervous system and many cardiovascular-acting drugs, which are used for rapid onset in critical care situations, as a premedication prior to surgery, or for the induction of anesthesia.

Further, additional stress is placed on the liver as it removes the excess drug from the bloodstream. This is particularly severe if the drug treatment has been occurring over an extended period of time. The liver may become overloaded with the drug's metabolite which then must be excreted. As a result, there is an increased risk of hepatic or renal disorders.

Another difficulty encountered in administering drugs orally is that dosages are prepared or determined for use with an “average” patient. Most drugs have widely varying effects on different patients. These effects depend upon patient habits, subtle genetic differences between patients, blood volumes, age, and numerous other known and unknown factors. Introducing a bolus of drug orally does not provide the ability to control the precise dose needed to obtain the desired effect, rather the dose is estimated in order to produce an average effect in an average patient. The result may be under dosing or overdosing a particular patient.

Under dosing a patient because of a low susceptibility to the drug fails to evoke the response sought by the physician. Overdosing the patient can result in dangerous depression of vital body functions, especially the heart and lungs. This can cause prolonged respiratory depression (necessitating mechanical ventilation after surgery), cardiac depression, and cardiac arrest.

In order to avoid some of the disadvantages of oral administration, injection is frequently used. Injecting a drug (generally intravenously or intramuscularly), results in rapid entry of the drug into the patient's bloodstream. In addition, this type of delivery avoids the removal of large quantities of the drug by the patient's liver. As a result, less total drug is usually needed compared to orally administered drugs. The drug instead becomes rapidly distributed to various portions of the patient's body before exposure to the liver.

Most patients, particularly children and geriatric adults, have an aversion to injections. In some patients, this aversion may be so pronounced as to make the use of injections a serious concern. Since intense psychological stress can exacerbate a patient's debilitated condition, it sometimes becomes undesirable to use injections where the patient is seriously ill or suffers from a debilitating condition or injury.

In addition, individual variations in susceptibility to the metabolism of various drugs (particularly drugs with central nervous system activity) are even more profound when utilizing the injection route. In many instances to prevent overdosing, it is the practice to inject a patient with a lower than average dose and then supplement the dose with additional injections as necessary. This “titration” makes necessary the use of repeated injections, which in turn greatly increases stress on the patient. Again, a precise dose cannot be administered to produce a precise effect because the patient's response varies widely depending on the specific characteristics of the individual patient.

One common approach to preparing a patient for surgery is to orally administer a sedative or anxiolytic. Although quick onset of sedation or anxiolysis has not always been a critical factor, it is more so now. Changing practices, including the increased use of outpatient units for day surgery and the pressures for cost containment in modern medicine, dictate the necessity for rapid onset of drug action and the use of an absolutely ideal dose in order to avoid increased costs of caring for patients with delayed recovery resulting from slightly overdosing with an anesthetic. Effective oral administration of premedication drugs with central nervous system activity (which cause a rapid onset of sedation and anxiolysis without producing excessive sedation) is often difficult to accomplish.

Some investigators have suggested that it may be possible to administer medication through the buccal mucosa or by sublingual administration. See, U.S. Pat. No. 4,671,953 to Stanley et al. entitled “METHODS AND COMPOSITIONS FOR NONINVASIVE ADMINISTRATION OF SEDATIVES, ANDROGENS, AND ANESTHETICS.” Such administration through the mucosal tissues of the mouth, pharynx, and esophagus of therapeutic drugs possesses a distinct usefulness. Administration of drugs by this route does not expose the drug to the gastric and intestinal digestive juices. In addition, the drugs largely bypass the liver on the first pass through the body, thereby avoiding additional metabolism and/or inactivation of the drug.

Generally the drugs which are administered by any of the methods described above have an unpleasant taste. As a result, in order to allow for buccal or sublingual administration through the oral mucosal tissues, it is also necessary to incorporate the drug into some type of pleasant tasting mass, such as a “candy” matrix.

In the manufacture of medicated candy products by existing methods, the therapeutic agent is added to a molten candy mass. The resultant mixture is then thoroughly mixed to ensure distribution of the drug within the molten candy mass. The mixture is then poured into a mold cavity while still molten and allowed to solidify into a solid mass. The hot candy mass may be poured into various molds, the size and shape of which may be determined as desired.

For effective application of the drug, the final candy product may contain the drug uniformly distributed throughout in order to ensure uniform levels of medication. Alternatively, for some applications, varying concentrations within known and controlled ranges may be desired to vary the rate of drug administration. Difficulties have been encountered, however, in attempting to blend solid drugs in a uniform or otherwise carefully controlled manner. Many drugs are insoluble, or only partially soluble, in one or more of the ingredients of the hard candy base. Thus, the resultant product is often found to be lacking in uniform or controlled distribution of the drug.

In addition, it is often found that when the temperature of the candy mass is increased in order to enable a more uniform distribution (generally to a temperature above approximately 230° C.), considerable decomposition of the drug may take place. While the extent of decomposition may vary, high temperatures are generally undesirable in the handling and processing of medications. Thus, the process of formation of the candy product may itself degrade and/or inactivate the therapeutic agent.

Key formulations, i.e. buffers, fillers, solubilizers, enhancers, etc., and the physical factors controlling dissolution, have not been optimized for oromucosal absorption.

It should also be noted that pH conditions within the mouth may tend to adversely affect the administration of certain lipophilic drugs by the mucosal administration route. It has been found that administration of drugs through the mucosal tissues generally occurs best when the drug is in the unionized form. Variations in pH affect the percentage of the drug which is unionized at a particular point in time. As a result, the pH conditions within the mouth can limit the effectiveness of certain drugs administered buccally or sublingually in that those conditions cause the drug to exist in the ionized form which is largely unavailable for transfer across the mucosal tissues.

Other potent drugs are substantially nonlipophilic and do not naturally permeate mucosal tissues. Hence it would be a significant advancement in the art of administering potent, fast-acting drugs, if suitable methods and compositions permitted both lipophilic and nonlipophilic drugs to be administered transmucosally in a controlled manner.

It would be a further significant advancement in the art to provide methods and compositions for incorporating drugs (including insoluble drugs) into a dissolvable matrix without heating the mixture to the point that degradation occurs. It would be a related advancement in the art to provide such a method which provided the capability of uniformly incorporating insoluble drugs into the dissolvable matrix.

### SUMMARY AND OBJECTS OF THE INVENTION

The present invention is directed to compositions and methods of manufacture for producing dissolvable or nondissolvable drug-containing dosage-forms for use in administering transmucosal potent, fast-acting drugs. The present invention is particularly useful in delivering central nervous system affecting drugs or medicaments to the body of a patient, such as various sedatives, analgesics, anxiolytics, amnestics, and anesthetics.

In use, the present invention provides for the administration of drugs through the mucosal tissue of the mouth, pharynx, and esophagus, thereby avoiding some of the problems of injection or oral administration. Employing the present invention, the drug may be introduced into the patient's bloodstream almost as fast as through injection, and much faster than using the oral administration route, while avoiding the negative aspects of both methods.

The term “potent” when used in describing drugs contained within the dosage forms described herein means a drug requiring a relatively small dose in the lozenge or tablet when compared with the size and/or weight of the dosage form. Potent drugs also include drugs used in a dosage form having a low ratio of active ingredients to excipient. Moreover, if the effective drug concentration is typically less than 100 ng/ml in blood, such a drug is also considered potent.

In one embodiment of the invention, a dissolvable drug-containing dosage-form for use in transmucosal delivery of a drug to a patient comprises a binding agent that is formed into a solid matrix dissolvable in the mouth of a patient. A pharmacologically effective dose of a central nervous system affecting drug that can be absorbed through mucosal tissues of the mouth, pharynx, and esophagus is dispersed throughout the solid matrix. A buffer or permeation enhancer may also be dispersed throughout the solid matrix along with the drug. The permeation enhancer may be capable of modifying the permeability of the mucosal tissues of the mouth, pharynx, and esophagus towards the drug in order to facilitate transmucosal absorption of the drug. A buffer alters the pH to increase absorption. When the solid matrix dissolves in the mouth of the patient, the pharmacologically effective dose of the drug is released for absorption through mucosal tissues of the mouth, pharynx, and esophagus of the patient.

A dissolvable binding agent formed into a solid matrix can be selected from a variety of materials having suitable binding properties for forming the matrix material. Carbohydrates that are dissolvable in the mouth of a patient such as various celluloses, starches, sugars, and derivatives thereof can be used as the binding agent. In addition, the binding agent can be selected from various fats, proteins, hydrocarbons, waxes, hydrogels, and dissolvable resins. The above binding agents can be used singly or in a variety of mixtures, depending on the desired characteristics of the matrix material.

In a method of making a dissolvable drug-containing dosage-form, a binding agent is selected that is dissolvable in the mouth of a patient, and a pharmacologically effective dose of a central nervous system affecting drug is added to the binding agent. A permeation enhancer is also added to the binding agent. The binding agent, drug, and permeation enhancer are mixed to form a moldable mixture in which the drug and permeation enhancer are dispersed throughout the binding agent. A solid matrix dissolvable in the mouth of a patient is then formed from the moldable mixture.

In forming the solid matrix, the mixture may be compressed, poured into a mold cavity, dehydrated, freeze dried, or otherwise formed into a suitable dosage form. Additional components may be incorporated into the moldable mixture before the solid matrix is formed such as flavors, dyes, mold releasing agents, and other additives. The drug can be dispersed substantially uniformly throughout the binding agent, can be contained within the binding agent, or can be adhered to the binding agent. In one preferred embodiment, the solid matrix is formed from a mixture of solid powder components that are compressed. The compressed powder dosage-form improves the shelf-life and stability of the drug.

The dissolvable dosage-form of the present invention provides the advantage of controlling the dissolution rate of the composition once it is administered to a patient. This can be accomplished in a number of ways. The dissolution rate may be modified chemically by including a hydrophobic agent to slow dissolution or a hydrophilic agent to enhance dissolution. The solubility of the selected binding material, e.g., carbohydrate, fat, protein, wax, etc., likewise affects the dissolution rate. Dissolution may also be controlled by the extent to which the mixture is mechanically compressed in producing the dosage-form. In addition, dissolution can be accomplished by varying the vigor with which the patient sucks on the dissolvable matrix.

In another embodiment of the invention, a nondissolvable drug-containing dosage-form comprises a drug containment matrix that is nondissolvable in the mouth of the patient, and a pharmacologically effective dose of a central nervous system affecting drug that can be absorbed through mucosal tissues of the mouth, pharynx, and esophagus. The drug is incorporated into the nondissolvable matrix which is configured to release the drug within the mouth of the patient for absorption through the mucosal tissues. The nondissolvable drug-containing dosage-form may also include a permeation enhancer to facilitate transmucosal absorption of the drug.

Drugs that are incorporated into the nondissolvable drug-containing dosage-form can be in a medicament medium, can be microencapsulated, or can be contained within sponge-like matrices or microsponges that are biologically inert and capable of entrapping a drug and then releasing the drug over time. Where the drug is incorporated into sponge-like matrices, the dosage-form may be designed to release the drug in response to pressure, either negative or positive, or other similar release trigger.

In a method of making a nondissolvable drug-containing dosage-form, a pharmacologically effective dose of a central nervous system affecting drug is selected along with a drug containment matrix that is nondissolvable in the mouth of a patient. The drug is then incorporated into the nondissolvable matrix.

In alternative embodiments of the nondissolvable dosage-form of the invention, a permeable membrane or screen-like barrier contains a drug as part of a medicament medium, a microencapsulated drug, or a drug within sponge-like matrices. The drug is retained within the permeable barrier under conditions outside the patient's mouth while being capable of permeating the barrier when the dosage-form is exposed to conditions of the mouth, pharynx, or esophagus.

For example, the drug can be an ingredient of a pharmaceutically acceptable carrier having a viscosity such that the drug will not permeate the permeable barrier outside the mouth of the patient, but the carrier has a change in viscosity such that the drug will permeate the permeable barrier when the dosage-form is exposed within the mouth of the patient. This change in viscosity can be due to salival contact with the carrier such that the drug will permeate the permeable barrier when the dosage-form is within the mouth of the patient. Alternatively, the temperature within the mouth of the patient can cause the viscosity of the carrier to be altered such that the drug will permeate the permeable barrier when the dosage-form is within the mouth of the patient.

A number of factors influence the drug administration rate of a nondissolvable dosage-form. For instance, incipient solubility, formulation of the drug (microencapsulated, microsponge), pore size and charge (electropotential) on a permeable barrier, and the force or vigor with which the patient sucks or squeezes the dosage-form affect the drug administration rate. In addition, the drug solvent (if the drug is in liquid form), i.e., water or oil affects the administration rate.

The present invention overcomes many of the problems of the prior art. For example, insoluble drugs can be added to the dissolvable matrixes of the invention without the necessity of attempting to dissolve the drug. In addition, high temperatures, which are generally required to form a molten candy matrix and which can cause degradation of some drugs, are avoided in methods of the present invention. Therefore, even drugs with relatively low melting points or those drugs which can experience decomposition below their melting points, can be incorporated into the compressed powder dosage-forms of the invention. A further advantage of the present invention is that flavoring problems are overcome in many cases. Flexibility in adding flavors is provided in that solubility of the components is not required in order to incorporate any particular flavor into the matrixes.

Buffering agents can also be added to the dosage forms of the invention in order to provide for maximum drug efficiency. Buffering agents are particularly important for those drugs that partially ionize within the pH range of the mouth, such as weak acid and weak base drugs. Therefore, if pH conditions can be adjusted to maximize the percentage of unionized drug available, the effectiveness of the drug is maximized. Buffering agents can be used that are capable of modifying the salival pH such that a majority of the drug remains unionized in order to facilitate transmucosal absorption of the drug.

A drug released from a dissolvable or nondissolvable drug containment matrix according to the present invention and administered through the oral mucosal tissues will quickly enter the patient's bloodstream through the veins which serve these tissues. Appropriate monitoring of the patient's reaction to the drugs which have an observable or monitorable effect will indicate when the drug has evoked a suitable response. The dosage-form may then be removed, or its rate of consumption may be modified in order to maintain the desired effect.

It will be appreciated that the ever present risk of overdosing a patient is substantially minimized through the use of the present invention. According to the present invention, the drug dose is given over a period of time rather than all at once, and the administration rate can be adjusted if it appears to be necessary. Once a sufficient drug response has been achieved, the patient can simply stop sucking or squeezing the dosage-form or the patient or medical professional can easily remove the dosage-form from the patient's mouth.

Relative drug permeability of the oromucosa has been characterized using model compounds for the sublingual, buccal, and gingival areas. The sublingual area generally includes the tissues beneath the tongue, the buccal area includes the tissues between the cheek and the upper molar gums, and the gingival area includes the tissues between the incisor gums and the upper lip. The three sites have different drug permeability, the sublingual area having the highest permeability and the gingival area having the lowest.

Because these areas have different permeability characteristics, a patient may select and, to some extent, manipulate the uptake of an oral transmucosally delivered drug by moving the dosage form, such as a freely-movable lozenge, from one area to another and by controlling the dissolution rate of the lozenge through active sucking. If the lozenge is freely movable in the patient's mouth, the patient can move the lozenge from an area of high permeability to an area of lower permeability with relative ease. Patients who regularly require treatments with the lozenge can effectively self titrate the lozenge dose through a process of trial and error. Over time, as the patient repeatedly uses the lozenge, the patient will develop a sensitivity to the effect of the drug depending upon its position and the amount of time the lozenge is located in that position. This may allow the patient to administer the drug more effectively.

The freely movable lozenge provides other advantages. Because the lozenge can be formulated with a permeation enhancer, extended contact between the permeation enhancer and a particular area of tissue in the oral cavity could increase the likelihood of irritation of the tissue. As a lozenge is moved about the mouth, the permeation enhancer spends less time in contact with one area, and the likelihood of irritation of a particular area of tissue due to extended contact with the permeation enhancer is reduced. Moreover, since the sublingual area is regarded as an area of high permeability, it is likely that a patient would prefer to maintain a lozenge in that position. However, having the lozenge in the sublingual area, particularly if the lozenge is fixed in that position, can make it difficult for the patient to carry on a conversation. With a freely movable lozenge, the patient can reposition the lozenge to a different part of the oral cavity where it would not impair the patient's speech. Likewise, other activities, such as chewing or drinking, may require the lozenge to be moved from one area of the mouth to another in order to facilitate the activity. The ability to move the lozenge without having to handle the lozenge is advantageous.

It is, therefore, an object of some embodiments of the present invention to provide dosage-forms that accomplish the noninvasive administration of a drug to a patient in order to rapidly induce a desired systemic effect.

It is another object of some embodiments of the present invention to provide methods and compositions for forming a dissolvable drug-containing matrix which avoid degradation of the drug, overcome problems related to insolubility of the various components in the dissolvable matrix, and provide a product which is not likely to crumble in the patient's mouth.

It is a further object of some embodiments of the present invention to provide methods and compositions for forming a nondissolvable drug-containing matrix which do not require heating the drug to the point that degradation occurs and which permit the use of stable drug forms.

Another object of some embodiments of the present invention is to provide suitable methods and compositions for the noninvasive transmucosal administration of both lipophilic and nonlipophilic drugs.

It is another object of some embodiments of the present invention to provide compositions which allow for precise control of the dosage and effect of the drug to be administered.

It is a further object of some embodiments of the present invention to provide methods for administering potent, fast-acting drugs in a precise dosage to obtain a precise effect in every patient. A related object of the present invention is to provide such methods that avoid the disadvantages of overdosing, under dosing, and the immediate metabolism encountered in the “first pass effect,” yet do not involve injection by needle into the patient.

These and other objects and features of the present invention will become more fully apparent from the following description and appended claims, or may be learned by the practice of the invention as set forth hereinafter.

### BRIEF DESCRIPTION OF THE DRAWINGS

In order to more fully understand the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1A is a cross-sectional view of a dosage-form within the scope of the present invention including a medicament medium within a permeable barrier;

FIG. 1B is a cross-sectional view of a dosage-form within the scope of the present invention including a plurality of microencapsulated drug particles within a permeable barrier;

FIG. 1C is a cross-sectional view of a dosage-form within the scope of the present invention including a plurality of drug-containing microsponges within a permeable barrier;

FIG. 2A is an elevational cross-sectional view which is a partial cut away of a dosage-form within the scope of the present invention including a plurality of drug-containing microsponges bound together with a binding material;

FIG. 2B is an elevational cross-sectional view of a dosage-form within the scope of the present invention including a plurality of microencapsulated drug particles bound together with a binding material;

FIG. 3 is an elevational cross-sectional view of another dosage-form within the scope of the present invention including a nondissolvable fibrous covering embedded with medicament particles; and

FIG. 4 is a cross-sectional plan view of the embodiment illustrated in FIG. 3.

### DETAILED DESCRIPTION OF THE INVENTION

The present invention is directed to drug-containing dosage-forms and methods of producing dosage-forms for use in the buccal, sublingual, pharyngeal, and esophageal transmucosal delivery of medicaments to a patient. The dosage-forms include an oral dissolvable drug containment matrix or vehicle capable of releasing one or more therapeutic agents or drugs for administration through the oral mucosal tissues. A drug can be noninvasively administered through the mucosal tissues of the mouth, pharynx, and esophagus as the patient sucks or squeezes the drug-containing dosage-form.

The term “lozenge” as used herein refers to a dosage-form that is an oral dissolvable or nondissolvable drug containment matrix capable of releasing one or more drugs. The term lozenge includes tablets, trouches, caplets. Other dosage forms contemplated as being included in the present invention are forms conveniently placed in the mouth of a patient in order to administer a potent drug through the oral mucosal tissues. A lozenge can be easily removed by the patient or caregiver when the desired effect is achieved. This provides a convenient dosage control mechanism through self-titration or moderation of the drug as the patient sucks or squeezes on the dosage-form.

The present invention overcomes several of the limitations encountered in the delivery of drugs either orally or by injection. One of the primary advantages of the present invention is the ability to introduce a drug to a patient in a “dose-to-effect” manner, in which the drug is administered to the patient until the precisely desired effect is obtained. Once the desired effect is obtained, the patient or the medical professional simply removes the dosage-form from the patient's mouth. This is different from prior art methods where a predetermined quantity of the drug is introduced into the patient all at once.

1. General Discussion of Compositions and Methods of Manufacture

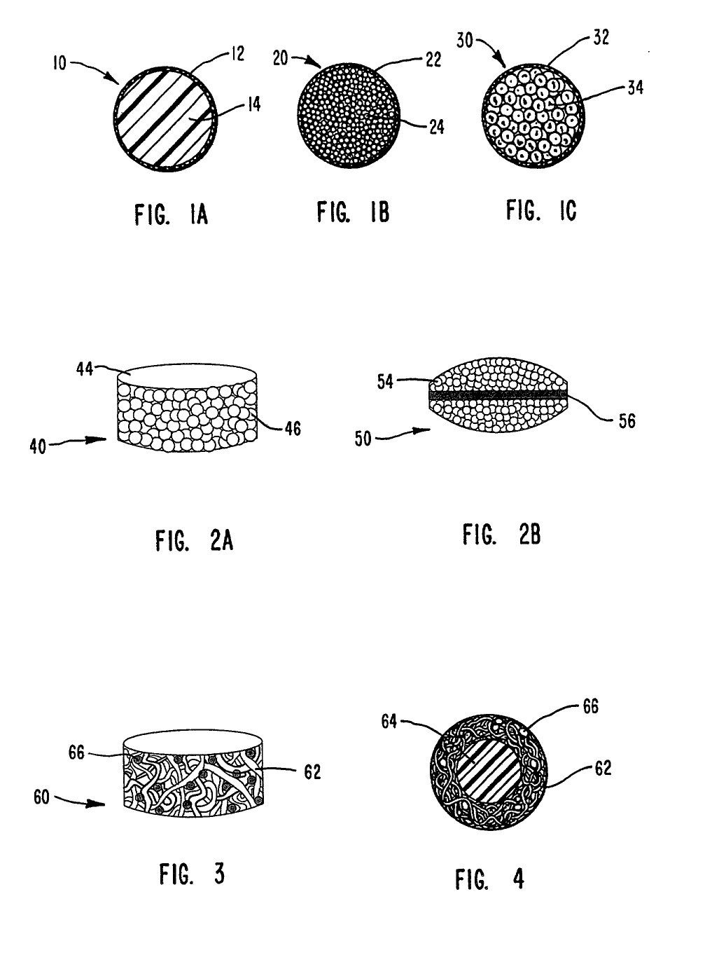

FIG. 1A shows how the present invention achieves the various advantages thereof in one embodiment. In this embodiment, a dosage-form 10 incorporates a drug or therapeutic agent into a drug containment matrix 14, such as by embedding the drug within the matrix. The drug may be incorporated into a variety of possible containment matrixes. For example, the drug may be incorporated into droplets coated with protective coating material 24, as shown in a dosage-form 20 in FIG. 1B; the drug may be microencapsulated into a sponge-like matrix or a microsponge 34, as shown in dosage-form 30 in FIG. 1C.

In addition, the drug may be contained within a permeable membrane or screen-like barrier shown at 12, 22, and 32, respectively, in FIGS. 1A, 1B, and 1C. Also, a biocompatible composition may be used to maintain the nondissolvable matrix in a preselected dosage-form shape. For example, FIGS. 2A and 2B show a biocompatible binding material or adhesive, 44 and 56, that may be used to adhere together a plurality of microencapsulated drug particles 54 or microsponges 46 into a preselected shape. In FIG. 2A, the binding material 44 comprises a coating that surrounds the drug. In FIG. 2B, the binding material 56 is a compressible binder.

The present invention overcomes many of the problems encountered generally in melting and incorporating drugs into a hard candy matrix. For example, in the present invention, by compressing the powders or liquids at room temperature, as opposed to the mixing of liquid components at elevated temperatures as in the prior art, the degradation of drugs, which often occurs at the elevated temperatures needed to produce a molten candy mass is avoided. This facilitates use of drugs having relatively low melting points, or those drugs which can experience decomposition at elevated temperatures below their melting points. The mixing can also be done at low temperatures. In this way, evaporation of any volatile ingredients is minimized and the “stickiness” of certain ingredients is reduced.

In addition, because solid powders or non-heated liquids are combined together, constituents which may be chemically incompatible when in a heated solution or suspension can be mixed. In forming medicated confections by known methods, severe problems are encountered in that the medication, flavorings, and other components may be insoluble when placed in the same heated liquid environment. When employing the method of the present invention for making a dissolvable dosage-form, there is no need to heat the mixture to a molten mass. As a result, heat degradation of the drug component is avoided while good mixing and a uniform product are provided. Thus, problems of chemical incompatibility between ingredients is eliminated in the present invention.

Once the desired constituents are thoroughly mixed, they may be formed into a solid dissolvable dosage-form. In other cases the constituents are wetted to form a slurry, dried, and then compressed (sometimes referred to as “slugging”) under relatively high forces to form a coherent dosage-form. Typically, compressive forces in the range from approximately 2,000 Newtons to approximately 5,000 Newtons are preferred, however, any force which is sufficient to compress the ingredients into a coherent, integrated mass could be used. As a result, the compressed powdered matrix is held together by physical means rather than by chemical means. The extent of the compressive forces used can be modified to vary the rate at which the dosage-form will dissolve in a patient's mouth. The greater the compressive forces that are used to form the dosage-form, the slower the dissolution of the matrix material in the mouth.

In other embodiments within the scope of the present invention, the desired constituents for a dissolvable dosage-form are formed into the dosage-form by dehydration, freeze drying (lyophilization), pouring into a mold, vapor deposition, or other known techniques in the art. In particular, wet granulation is the preferred mode to ensure uniform distribution of the mixed materials.

Since the present invention teaches the use of different dissolvable matrix materials which can be compressed, poured, dried, or otherwise formed into a solid dosage-form, virtually any desired type of mold can be used for the formation of the dosage-form. The active ingredients added to the dissolvable matrix materials may be in solid form, liquid form, or microencapsulated. In some embodiments within the scope of the present invention, is specific confectionery components are combined in order to form an integral solid mass that is dissolvable. These components may include, for example, compressible confectioner's sugar, sorbitol, mannitol, and maltodextrin.

The present invention provides a great deal of flexibility in the making of an appropriate drug-containing dosage-form, with the quantity of drug contained in any dosage-form being variable within a wide range. In addition, the present dosage-form facilitates the transmucosal absorption of a variety of therapeutic agents, and allows for verifiable transfer of a medication to the patient. For instance, the medication may be bound to a dye such that loss of color indicates transfer of the medication to the patient.

The present invention provides the capability of providing a good tasting or palatable medication. With many drugs, it has previously been extremely difficult to provide a good tasting medicine because of the extreme bitterness or other unpleasant taste of many drugs. The embodiments of the present invention that employ microencapsulation and microsponge technologies are able to help mask the unpleasant taste of many drugs. In addition, favorable taste characteristics can be accomplished by adding various flavors, sweeteners, and the like. Since the components are combined as solids or liquids (or even liquids that are slowly released from microsponges), problems associated with combining flavoring components insoluble in a molten candy mass are avoided.

The present invention allows for use of the free acid form or the free base form of certain drugs. In addition, some embodiments employ buffering agents to moderate pH. Generally, buffering agents are more important when hydrophilic drugs are used because those drugs usually have lower mucosal permeability and dissolve more readily in saliva within the mouth. In addition, pH greatly affects absorption and can be useful in creating specific pharmacokinetic profiles. For example, increasing absorption will result in faster onset and achievement of the desired indication, but will not allow much drug to pass to the gastrointestinal area. This results in a steep profile with short duration. For longer duration efficacy, a pH allowing lower absorption transmucosally could be employed. This would allow more drug to pass to the gastrointenstinal tract providing for longer term absorption.

Unlike administration of drugs orally or by injection, the present dosage-form can be removed from the mouth of a patient to easily halt further administration of the drug.

2. Compositional Components and Materials

In order to prepare a desirable drug-containing dissolvable matrix for formation into a dosage-form, it is generally necessary to combine several general types of components. These components include the types of components used to prepare typical confections, the desired drug, and other chemically active ingredients such as buffering agents, permeation enhancers, and the like. The types of components that can be used generally fall into the following categories:

(1) binding agent,

(2) sweeteners,

(3) flavorings and flavor enhancers,

(4) releasing agents,

(5) buffers, and/or permeation enhancers,

(6) one or more therapeutic agents,

(7) bulking agents, and

(8) solubilizers.

As mentioned above, it is preferred that these components each be provided in a form which facilitates mixing, such as a dry powder or wet granulation. This provides for convenient combination of the ingredients, even if they happen to be insoluble or otherwise chemically incompatible.

The binding agent formed into a solid matrix can be selected from a variety of materials having suitable binding properties for forming the matrix material. The binding agent can be any water-soluble or dispersible material that is acceptable for pharmaceutical uses and inert towards the active ingredients. Carbohydrates that are dissolvable in the mouth of a patient such as various celluloses, starches, sugars, and derivatives thereof can be used as the binding agent. For example, various cellulose derivatives can be used such as microcrystalline cellulose, carboxymethyl cellulose, etc., as well as water-dispersible starch derivatives, and various compressible sugars. In addition, the binding agent can be selected form various fats, proteins such as gelatin, hydrocarbons, waxes, hydrogels, and dissolvable resins such as natural or synthetic resins (e.g., gum arabic, xanthan gum, etc.). The above binding agents can be used singly or in a variety of mixtures, depending on the desired characteristics of the matrix material.

The components may be a releasable or slowly releasable liquid ingredient of the medicament medium or the components may be incorporated within a sponge-like matrix or microencapsulated.

A wide range of flavors are available for preparing good tasting and desirable medications within the scope of the present invention. These may be required in order to mask the unpleasant taste of the drug. Flavorings may be combined, as desired, to produce a particular flavor mix which is compatible with a particular medication. Some of the confectioner's flavorings which are useful in the context of the present invention include artificial vanilla, vanilla cream, mint, cherry, berry, spearmint, grape, coconut, chocolate, menthol, licorice, lemon, and butterscotch.

Each of these flavorings is obtainable in a concentrated powdered form. Other flavorings known in the confectionery arts may also be acceptable because of the ease of combining the ingredients of the present invention. Any number of flavorings may be combined in any desired ratio in order to produce the specific desired taste characteristics required for any particular application. For example, flavor combinations may be varied in order to be compatible with the flavor characteristics of any specific drug.

In order to provide a palatable medication, sweeteners are preferably added to the composition. Sweeteners which are presently preferred include aspartame (NutraSweet®) and compressible confectioner's sugar. Other sweeteners, such as fructose, sorbitol, mannitol, xylitol, cyclamates, acesulfame K, thaumatin, sucralose, alitame, PS99/PS100, glycyrrhizin, monellin, stevioside, miraculin, or L-sugars may also be acceptable for use within the scope of the present invention. A sweetener or combination of sweeteners can be used which is compatible with the selected drug and the other components such that a palatable dosage-form is produced.

Maltodextrin and cyclodextran may also be added to provide a better tasting composition. Maltodextrin and cyclodextran are generally employed in order to dissipate unpleasant flavors (such as the bitter taste of most drugs) within the composition. In addition, maltodextrin is a highly compressible powder which facilitates the formation of compressible dosage-forms within the scope of the present invention.

For some applications, it may be desirable to add a flavor enhancer to the composition in order to achieve a good tasting product. Flavor enhancers provide a more pleasant sensation in the patient's mouth while the dosage-form is residing therein. Flavor enhancers within the scope of the present invention include materials such as ribotide (a nucleotide) and monosodium glutamate (“msg”).

Appropriate changes in flavoring ingredients can be made to mask or optimize flavor perception in order to achieve ultimate acceptance of the dosage-form by the desired patient group, be it adult, juvenile, pediatric, or neonate.

Added to the dissolvable drug containment matrix will be the appropriate therapeutic agent or drug. Various types of drugs are easily incorporated into the matrix compositions of the present invention. These preferably include therapeutic agents which in particular affect the central nervous system in the body of a patient and cause fast systemic effects such as sedation, anxiolysis, analgesia, amnesia, and anesthesia.

As will be discussed in more detail below, it may also be desirable to include buffering agents within the compositions of the invention. Buffering agents provide a favorable pH environment to stabilize the drug in the formulation and to optimize absorption of medicaments across the mucosal tissues of the mouth, pharynx, or esophagus. It will be appreciated that drugs in the unionized form are more readily transported across the mucosal membrane. Buffering agents incorporated within the composition can be used to effect a pH change in the salival environment of the mouth in order to favor the existence of the unionized form of the drug which then more readily moves through the mucosal tissues.

In addition, appropriate pH adjustment can aid in producing a more palatable product with drugs which are either severely acidic (and thus sour) or severely basic (and thus bitter). As a result, a buffer system such as citric acid/sodium citrate has been found to be desirable for addition into the dissolvable matrix. A phosphate or carbonate buffer system may also be used. Tromethamine (TRIS) can also be used to buffer the formulation to reduce the salty or brackish taste associated with buffers in the higher pH range.

A suitable permeation enhancer capable of improving the drug permeability across the mucosal membrane may also be included in the composition. The permeability of both lipophilic and nonlipophilic drugs may be improved by using suitable permeation enhancers. Permeation enhancers are particularly important when nonlipophilic drugs are used, but may be valuable for certain lipophilic drugs as well. Examples of typical permeation enhancers which may be used within the scope of the present invention are discussed below.

It will be appreciated that miscellaneous other additive ingredients may also be used. For example, in order to produce a desirable color for the end product, artificial colorings may be added to the compositions of the invention. The flavorings described above are generally a white powder, as are the other major components. Therefore, additional coloring is necessary if a colored end product is desired. Coloring may also be important as a code to indicate the type and concentration of drug contained within a particular dissolvable matrix. Any type of color known to be “FD&C” certified may be used to provide coloring to the product.

In certain embodiments, it may also be desirable to add a lubricating or releasing agent in order to facilitate the release of the dosage-form from a manufacturing mold. Such agents may also provide a certain amount of waterproofing and chemically modified dissolution rate control. The lubricating or releasing agents may include hydrophilic agents such as lactose which act to enhance dissolution of the dosage-form, or may include hydrophobic agents which act to inhibit dissolution such as compritol 888 (glyceryl behenate), calcium stearate, magnesium stearate, and the like, including mixtures thereof.

Lubricating agents and surfactants are useful in those embodiments wherein a powder mixture is funneled into a chute during manufacture. Lubricating agents and surfactants improve product flow and avoid static electricity charge buildup within the formulation which may cause the ingredients to separate due to electrostatic forces.

Lactose can also be employed to provide filling and bulk to the dosage-form as well as to enhance dissolution of the dosage-form, and gelatin may also be used as a filling and bulking agent. Other filling and bulking agents known to those skilled in the art may also be used.

The dissolvable dosage-form of the present invention can be formulated to control the dissolution rate of the composition once it is administered to a patient in a number of ways. As discussed above, the dissolution rate may be modified chemically by including a hydrophobic agent to slow dissolution or a hydrophilic agent to enhance dissolution. The solubility of the selected binding material likewise affects the dissolution rate. Dissolution may also be controlled by the extent to which the mixture is mechanically compressed in producing the dosage-form. In addition, dissolution can be controlled by varying the vigor with which the patient sucks on the dissolvable matrix.

In other embodiments of the invention, a biocompatible adhesive may be incorporated into the dissolvable matrix material such that a dosage-form of the invention can be placed in a preselected position within the mouth of the patient, such as adhered to the side of the mouth. This has the advantage of allowing the dosage-form to remain in one place in a convenient location in the mouth of a patient until such time as the dosage-form is removed, giving the patient freedom to use his mouth for talking, eating, drinking, etc. while the dosage-form is positioned therein. In addition, a more steady dose of the drug can be maintained since the dosage-form remains in one place in the mouth of the patient.

Microencapsulated drugs, mentioned above in reference to FIGS. 1B and 2B, are drug particles or droplets which have been coated with a protective coating material. Typical coating materials include fats, waxes, triglycerides, fatty acids, fatty alcohols, ethoxylated fatty acids and alcohols, stearates, sugars, poly(ethylene glycol), certain metals, gums, hydrocolloids, latexes, and various polymer-based formulations such as polyethylene, ethyl cellulose, ethylene-vinyl acetate, ethylene-acrylic acid, polyamides, and some enteric polymers.

The protective coating material of microencapsulated drugs prevents drug degradation by moisture, retards oxidation of the drug, decreases evaporation and sublimation, protects the drug from reaction with other ingredients, and masks the unpleasant taste of some drugs. Typical drug microencapsulation techniques useful in the present invention are known to those skilled in the art.

Sponge-like matrixes or microsponges, mentioned above in reference to FIGS. 1C and 2A, are devices capable of entrapping a medicament and then releasing the medicament within the mouth of the patient over time in response to pressure exerted on the sponge-like matrix by the mouth of the patient. These sponge-like matrixes are biologically inert, non-irritating, non-mutagenic, non-allergenic, non-toxic, and non-biodegradable.

Like true sponges, the sponge-like matrixes or microsponges contain a myriad of interconnecting voids within a non-collapsible structure with a large porous surface. The size of the sponge-like matrix as well as the number and size of the internal pore structure can be varied depending on the medicament size and viscosity.

The medicament is released from the sponge-like matrix in response to a suitable “trigger.” For example, rubbing or pressing the sponge-like matrix, elevating the temperature of the matrix (as within the patient's mouth vis-a-vis ambient temperature), or introducing suitable solvents such as saliva can cause a controlled release of the medicament. Pressure may also be used to release the drug from the sponge-like matrixes. Squeezing and sucking a dosage-form containing the sponge-like matrixes saturated with the medicament will release the medicament.

The use of encapsulation and microsponges allows the rate of absorption to be controlled. For example, if longer efficacy is desired, then more drug should be absorbed in the gastrointenstinal tract instead of transmucosally. By providing more drug in the microencapsulated state, the pharmacokinetic profile may be flattened and elongated.

FIG. 2B shows an embodiment of the present invention having microencapsulated drug particles retained within a compressed powder dosage-form. The microencapsulated drug particles 54 are compressed together with a compressible binder 56, such as compressible sugar and/or other ingredients as described above. FIG. 2A illustrates another drug-containing vehicle, microsponges, that may also be suitably retained within dosage-forms made with a dissolvable matrix material such as a compressible binder.

In another embodiment of the invention, shown in FIGS. 3 and 4, the drug may be held within nondissolvable containment vehicles capable of releasing the drug for transmucosal administration. In the depicted embodiment, a dosage-form 60 comprises a nondissolvable matrix 62 capable of entrapping the drug and then releasing the drug over time. In the particular embodiment shown, matrix 62 comprises a nondissolvable fibrous covering that is embedded with medicament particles 66.

Nondissolvable drug-containing dosage-forms comprise a drug containment matrix that is nondissolvable in the mouth of the patient, and a pharmacologically effective dose of a central nervous system affecting drug that can be absorbed through mucosal tissues of the mouth, pharynx, and esophagus. The drug is incorporated into the nondissolvable matrix which is configured to release the drug within the mouth of the patient for absorption through the mucosal tissues. The nondissolvable drug-containing dosage-form may also include a permeation enhancer to facilitate transmucosal absorption of the drug.

Drugs that are incorporated into the nondissolvable drug-containing dosage-form can be in a medicament medium, can be microencapsulated, or can be contained within sponge-like matrices or microsponges that are biologically inert and capable of entrapping a drug and then releasing the drug over time. Where the drug is incorporated into sponge-like matrices, is the dosage-form may be designed to release the drug in response to pressure, either negative or positive, or other similar release trigger.

In a method of making a nondissolvable drug-containing dosage-form, a pharmacologically effective dose of a central nervous system affecting drug is selected along with a drug containment matrix that is nondissolvable in the mouth of a patient. The drug is then incorporated into the nondissolvable matrix.

In alternative embodiments of the nondissolvable dosage-form of the invention, a permeable membrane or screen-like barrier contains a drug as part of a medicament medium, a microencapsulated drug, or a drug within sponge-like matrices. The drug is retained within the permeable barrier under conditions outside the patient's mouth while being capable of permeating the barrier when the dosage-form is exposed to conditions of the mouth, pharynx, or esophagus.

For example, the drug can be an ingredient of a pharmaceutically acceptable carrier having a viscosity such that the drug will not permeate the permeable barrier outside the mouth of the patient, but the carrier has a change in viscosity such that the drug will permeate the permeable barrier when the dosage-form is exposed within the mouth of the patient. This change in viscosity can be due to salival contact with the carrier such that the drug will permeate the permeable barrier when the dosage-form is within the mouth of the patient. Alternatively, the temperature within the mouth of the patient can cause the viscosity of the carrier to be altered such that the drug will permeate the permeable barrier when the dosage-form is within the mouth of the patient.

A number of factors influence the drug administration rate of a nondissolvable dosage-form. For instance, incipient solubility, formulation of the drug (microencapsulated, microsponge), pore size and charge (electropotential) on a permeable barrier, and the force or vigor with which the patient sucks or squeezes the dosage-form affect the drug administration rate. In addition, the drug solvent (if the drug is in liquid form), i.e., water or oil affects the administration rate.

It is important that the nondissolvable dosage-form configuration be biocompatible and capable of releasing the drug for absorption through the patient's mucosal tissues. In addition, the configuration should preferably have a structure, shape, and texture that is palatable to the patient.

3. Therapeutic Agents

In order for the present invention to operate effectively, it is necessary that the therapeutic agent or drug incorporated within the dissolvable or nondissolvable drug containment matrix be capable of permeating the mucosal membrane either alone or by suitable adjustments in the environmental pH, by other chemical modification, or in combination with a suitable permeation enhancer. In some embodiments, the therapeutic agent may be microencapsulated or incorporated into microsponges as discussed above.

The present invention has particular applicability to a variety of drugs affecting the central nervous system (CNS), such as various sedative, anxiolytic, amnestic, analgesic, and anesthetic agents. For example, the compositions of the present invention may include opiate agonists (narcotic analgesics) such as fentanyl, sufentanil, lofentanil, carfentanil, alfentanil, codeine, and morphine; opiate antagonists such as naloxone, and nalmefene; agonist-antagonist agents such as buprenorphine, and nalbuphine; anesthetics such as phencyclidine, ketamine, propanidid, propofol, and thiamylal; butyrophenones such as droperidol and haloperidol; benzodiazepines such as diazepam, lorazepam, midazolam, triazolam, oxazolam, and oxazepam; GABA stimulators such as etomidate; and barbiturates such as thiopental, methohexital, pentobarbital, and hexobarbital; cannabinoids such as Δ⁹ THC, CP 55,940, WIN 55,212, and levonantradol. It will be appreciated that these as well as many other drugs may be utilized within the scope of the present invention either singly or in various combinations.

For purposes of example, Table 2 lists some of the CNS-acting drugs that are suitable for incorporation into the dosage-forms of the invention, including presently contemplated ranges of dosages for the listed drugs.

TABLE 2

| Generic Drug | Drug Class | Dose Range | |
|---|---|---|---|
| alfentanil | opioid agonist | 0.5-50 | mg |
| sufentanil | opioid agonist | 5-500 | μg |
| lofentanil | opioid agonist | 0.1-100 | μg |
| carfentanil | opioid agonist | 0.2-100 | μg |
| naloxone | opioid antagonist | 0.05-5 | mg |
| nalbuphine | opioid agonist-antagonist | 1-50 | mg |
| diazepam | benzodiazepine | 1-40 | mg |
| lorazepam | benzodiazepine | 1-4 | mg |
| midazolam | benzodiazepine | 0.5-25 | mg |
| oxazepam | benzodiazepine | 5-40 | mg |
| triazolam | benzodiazepine | 250-1000 | mg |
| droperidol | butyrophenone | 1-20 | mg |
| haloperidol | butyrophenone | 0.5-10 | mg |
| propanidid | anesthetic | 1-10 | mg |
| propofol | anesthetic | 3-50 | mg |
| ketamine | anesthetic | 5-300 | mg |
| etomidate | GABA stimulator | 5-60 | mg |
| methohexital | barbiturate | 10-500 | mg |
| pentobarbital | barbiturate | 50-200 | mg |
| Δ⁹ THC | cannabinoid | 1-50 | mg |
| levonantradol | cannabinoid | 1-10 | mg |
| thiamylal | barbiturate | 10-500 | mg |
| thiopental | barbiturate | 50-500 | mg |

The present invention allows drugs to be incorporated within the dissolvable or nondissolvable drug containment matrix which would otherwise be insoluble, unpleasant tasting, or have other undesirable characteristics. This capability is provided by the various formation techniques of the dosage-form. The present invention also allows lipophilic as well as nonlipophilic drugs to be utilized. In addition, efficient delivery of the drug is facilitated while at the same time drug degradation is avoided. The drug can also be administered in a dose-to-effect manner so that the drug effect produced is precisely controlled.

Fentanyl is one presently preferred drug for use in the dissolvable dosage-form of the present invention. There is an extremely wide variation in metabolism and subjective experience of pain from individual to individual. Thus, it will be readily appreciated how difficult it is to administer a drug such as fentanyl to any particular individual with any confidence that such an individual will receive an appropriate dose. By using the dosage-form of the present invention, a person can be given a lozenge containing a drug such as fentanyl until the appropriate effect is achieved. A person having a high susceptibility to the drug might not finish even one lozenge, while a person having a low susceptibility to the drug might be given a second lozenge to achieve a desired effect.

The present invention allows a much higher percent of potent drugs to enter the bloodstream through transmucosal administration. Precise control over the dosage and effect of the drug is achieved through transmucosal administration of the drug by having a patient suck on a drug-containing dosage-form until the precise dosage and effect is obtained.

The dosage-forms of the present invention are particularly advantageous in oncology in that cancer patients can suck on a lozenge of the invention to reduce pain as needed. Cancer patients experience what is called “breakthrough” pain which results when the pain breaks through the pain threshold established by the around the clock pain medication. Since the dosage-form or lozenge of the invention allows the dosage of the drug to be moderated by the sucking action performed by the patient, the patient can aggressively suck the lozenge at the onset of breakthrough pain in order to decrease the pain.

4. Examples of the Present Invention

The following examples are given to illustrate various embodiments which have been made or may be made in accordance with the present invention. These examples are given by way of example only, and it is to be understood that the following examples are not comprehensive or exhaustive of the many types of embodiments of the present invention which can be prepared in accordance with the present invention.

### EXAMPLE 1

In this example, methohexital is incorporated into a dissolvable matrix form. Methohexital is a known potent lipophilic drug useful as an anxiolytic, sedative and for anesthetizing a patient. Its high potency and lipophilicity makes it an excellent drug for transmucosal administration in accordance with the present invention. Compressible sugar is selected as the dissolvable matrix material.

A suitable mixture is prepared by combining the following ingredients:

| Ingredient | wt % | grams |
|---|---|---|
| citric acid | 1% | 0.2 |
| ribotide | 2% | 0.4 |
| compritol 888 | 2% | 0.4 |
| aspartame | 2% | 0.4 |
| vanilla microcaps | 5% | 1.0 |
| vanilla cream microcaps | 5% | 1.0 |
| wild cherry microcaps | 3% | 0.6 |
| peppermint microcaps | 3% | 0.6 |
| compressible sugar | 20% | 4.0 |
| methohexital sodium | 25% | 5.0 |
| maltodextrin | 32% | 6.4 |
| | 100% | 20 |

The ingredients are combined in a mixer in such a fashion as to ensure a uniform distribution of all ingredients within the mixture. Aliquots of 2 grams each are then compressed using a force sufficient to provide a final volume of 2 cubic centimeters. The procedure results in the preparation of 10 oral transmucosal dosage-forms, each containing 0.5 grams of methohexital.

### EXAMPLE 2

In this example, methohexital is incorporated into a dissolvable matrix form. Gelatin is selected as the dissolvable matrix material. A suitable mixture is prepared by combining the following ingredients:

| Ingredient | wt % | grams |
|---|---|---|
| citric acid | 1% | 0.2 |
| ribotide | 2% | 0.4 |
| compritol 888 | 2% | 0.4 |
| aspartame | 2% | 0.4 |
| vanilla microcaps | 5% | 1.0 |
| vanilla cream microcaps | 5% | 1.0 |
| wild cherry microcaps | 3% | 0.6 |
| peppermint microcaps | 3% | 0.6 |
| methohexital sodium | 25% | 5.0 |
| gelatin | 52% | 10.4 |
| | 100% | 20 |

The ingredients are combined in a mixer in such a fashion as to ensure a uniform distribution of all ingredients within the mixture. Aliquots of 2 grams each are then formed by dehydration. The procedure results in the preparation of 10 oral transmucosal dosage-forms, each containing 0.5 grams of methohexital.

It will be appreciated that similar dosage-forms may be produced using other dissolvable matrix materials such as fats, waxes (natural or synthetic), proteins, hydrogels, dissolvable resins, or other suitable dissolvable matrix materials.

### EXAMPLE 3

In this example, triazolam is incorporated into a compressed dosage-form. Triazolam is a known potent lipophilic drug useful as an anxiolytic, amnestic, and for sedating a patient. Its high potency and lipophilicity makes it an excellent drug for transmucosal administration in accordance with the present invention.

A suitable mixture is prepared by combining the following ingredients:

| Ingredient | wt % | grams |
|---|---|---|
| triazolam | 0.05% | 0.01 |
| citric acid | 1% | 0.2 |
| ribotide | 2% | 0.4 |
| compritol 888 | 2% | 0.4 |
| aspartame | 2% | 0.4 |
| vanilla microcaps | 5% | 1.0 |
| vanilla cream microcaps | 5% | 1.0 |
| wild cherry microcaps | 3% | 0.6 |
| peppermint microcaps | 3% | 0.6 |
| compressible sugar | 25.65% | 5.13 |
| maltodextrin | 51.3% | 10.26 |
| | 100% | 20.0 |

The ingredients are combined in a mixer in such a fashion as to ensure a uniform distribution of all ingredients within the mixture. Aliquots of 2 grams each are then compressed using a force sufficient to provide a final volume of 2 cubic centimeters. The procedure results in the preparation of 10 oral transmucosal dosage-forms, each containing 1.0 milligrams of triazolam.

### EXAMPLE 4

The same procedure described with reference to Example 1 is used except that fentanyl is used in place of methohexital. Fentanyl is a potent lipophilic drug useful for sedating or anesthetizing a patient or for treating breakthrough pain. The high potency and lipophilicity of fentanyl make it an excellent drug for transmucosal administration in accordance with the present invention.

### EXAMPLE 5

The procedure of this example illustrates the use of a sedative-containing lozenge in accordance with the present invention to prepare a child for outpatient surgery. Outpatient surgery has become increasingly accepted as a cost-saving approach to many surgical procedures. Unfortunately, this approach does not give the physician and hospital staff much opportunity to place the patient at ease or let the patient become accustomed to his or her surroundings. It is, of course, desirable to make the outpatient visit as pleasant as possible. An important goal of the physician and staff is to minimize stress and discomfort while treating a patient's disease or condition. A relaxed and cooperative patient is also easier to treat.

Use of a sedative-containing lozenge in accordance with the present invention can do much to assist a patient in this situation through a very stressful period. Rather than give an oral medication, which is slow to act and uncertain in response, and rather than give an injection, which immediately makes a child distrustful and even more upset, the child is offered a lozenge.

The child's tension drops immediately as he or she turns their attention to the pleasant task of sucking the lozenge. Then, the calming influence of the sedative begins to take effect. Although a typical lozenge contains a drug dosage strong enough for children having even a low susceptibility to the drug, a physician or staff member will readily recognize the point where the child has received a suitable dose. A lozenge adapted for use in sedating a child might advantageously contain about 0.1 to 1 milligram of fentanyl.

### EXAMPLE 6

In the procedure of this example, an adult patient is given a drug-containing lozenge in order to exert sedative and anesthetic effects. Sufentanil in a lozenge dose of 50 micrograms is used. The adult patient is permitted to take a sufficient dose of medication so that he or she falls asleep. The drug-containing lozenge is either swallowed or removed and discarded.

### EXAMPLE 7

In the procedure of this example, an adult is given a drug-containing lozenge in order to exert sedative and anesthetic effects similar to the situation in Example 8. However, in this example, methohexital in a lozenge dose of 20 micrograms is used. The adult is permitted to take a sufficient dose of medication so that he or she falls asleep. Prior to falling asleep, the drug-containing lozenge is either swallowed or removed and discarded.

### EXAMPLE 8

In this example, the procedure of Example 7 is used except that the lozenge contains 0.5 micrograms of lofentanil. Since this drug is well suited for analgesic use, the patient has control over the amount of pain experienced.

From the foregoing, it will be appreciated that the present invention allows great flexibility and permits physician control on a case-by-case basis with respect to the dose given to a particular patient, and the rate at which that dose is given.

The use of a drug-containing lozenge for administration of sedatives, analgesics, or anesthetic agents is much faster acting than oral administration, and also avoids unacceptable loss of drug on a first pass through the liver before systemic distribution. Further, the use of a lozenge in accordance with the present invention provides for a relatively level drug plasma concentration, which is preferable when dealing with sedatives and analgesics.

In addition, a physician can easily monitor a patient's condition to ensure the patient receives a dose adequate to evoke a desired state of sedation or analgesia. If necessary, the physician can instruct the patient to alter the aggressiveness with which the patient sucks the lozenge, or can take the lozenge from the patient.

A patient can also self-administer a suitable analgesic using a lozenge in accordance with the present invention. Thus, a patient can place an analgesic-containing lozenge passively in his or her mouth for continuous low level administration of a drug, or can suck the lozenge from time to time as needed to reduce the pain.

Using a lozenge as the dosage-form in this instance is useful in that non-prescription lozenges are commonly used for a wide variety of purposes such as, for example, relieving throat pain and freshening breath. Accordingly, a patient may use a lozenge according to the present invention to control pain as described above while in the company of other people without drawing unwanted attention to the fact that the patient is receiving medication. Use of a lozenge will not attract attention or cause comment in most any situation, even a professional situation, whereas use of another, more evident, dosage-form such as an inhaler would raise questions.

It will be appreciated that the controlled transmucosal delivery of a potent drug from a lozenge dosage form overcomes a number of the previously cited limitations of prior art. Use of a potent drug in a lozenge form as defined herein requires a relatively low dose to size ratio such that the amount of drug within the lozenge is relatively small such that the bulk of the dosage form is not made up of the therapeutic agent. Uniform distribution of the drug throughout the dosage form can be obtained through direct compression of the components of the dosage form. Through this process even minute physical quantities of the active ingredient can be uniformly distributed throughout the dosage form.

With a potent drug uniformly distributed throughout the lozenge dosage form, the lozenge can be administered into the patient's mouth where the potent drug is thereby released and absorbed into the mucosal tissues in a slow and controlled process. For example, a potent drug such as fentanyl can be administered by a lozenge where the fentanyl dose is relatively small in comparison to the size of the lozenge, but where the fentanyl is uniformly distributed throughout the lozenge. The fentanyl can be administered to the patient by placing the lozenge into the patient's mouth where the lozenge releases small amounts of the uniformly distributed fentanyl into the patient's mouth where the fentanyl is absorbed through the mucosal tissues. For example, a 2 gram lozenge may contain 200 micrograms of fentanyl.

The dose to size ratio of the therapeutic agent to the lozenge allows a slow and more controlled release of the therapeutic agent. This permits the patient or caregiver time to assess the effects of the drug as it is absorbed through the mucosal tissues and enters the systemic circulation. Where a patient shows signs of overdose the patient or caregiver can remove the potent lozenge thus preventing harmful overdose. The lozenge provides two advantages over other orally administered dosage forms. In some circumstances patients may be self conscious about using a dosage form which makes it apparent to other persons that the patient is taking medication. But because the lozenge is entirely located within the mouth of the patient it may be less obvious and go unnoticed by third parties when the patient is taking medication. The size of the lozenge is sufficiently large however to allow the patient to easily remove the lozenge from the mouth when the patient or caregiver determines that the administered dose is sufficient. The use of a potent drug in a lozenge also overcomes limitations of oral or transdermal administration of drugs. As mentioned above, the injection of a drug provides for immediate absorption into the systemic system and thus is fast acting, however the injection provides no control over the rate at which the drug is taken up into the system, since delivery is immediate. On the other hand, oral ingestion of medication may provide a more controlled release rate from the dosage form as the dissolution of the dosage form takes place within the gastrointestinal system. However, there is significant delay between the absorption into the gastrointestinal circulatory system and the eventual dispersion of therapeutic agent throughout the general circulatory system. Thus, the present invention combining a potent drug with a lozenge dosage form provides for a controlled release rate from the dosage form while still providing fast and effective absorption transmucosally into the systemic circulation.

### EXAMPLE 9

Another example of the present invention provides for effective transmucosal delivery of potent lipophilic drugs such as opioids. It is understood that the treatment of physical ailments through the use of opiates such as morphine, codeine and fentanyl can be dangerous to the patient. Where the absorption of such therapeutic agents is not entirely predictable, it is anticipated that the use of a lozenge containing such a therapeutic agent will be used only when supervised such as in a hospital or home hospice care.

When using the present invention, as disclosed in this example a care giver or a patient with health care supervision, will administer the lozenge containing the opioid allowing the therapeutic agent to be absorbed transmucosally into the systemic circulation. The caregiver individually or with the assistance of the patient will then evaluate the reaction of the patient to the drug to determine whether the drug is having a desired effect. Supervised use of the lozenge containing the opioid allows the patient to obtain the desired effects of the therapeutic agent, through a delivery system that is less threatening and faster acting than other systems, but which through the supervised use of the lozenge, prevents overdose, underdose or abuse of the therapeutic agent.

### EXAMPLE 10

Use of a potent drug in combination with a lozenge is particularly pertinent as applied to the treatment of cancer. Cancer patients undergoing aggressive treatments such as radiation and chemotherapy often experience severe and chronic pain. To alleviate the pain associated with such treatments cancer patients are often given strong analgesics such as fentanyl to reduce the pain. However after prolonged use of fentanyl, the pain killing effect of the original dose may subside, leaving the patient to cope with the pain without the other medication. Once the patient has become more tolerant to the effects of the drug, it is difficult for doctors to prescribe a particular dose since it is uncertain what dose will be effective at reducing the pain. Increasing the dosage under such circumstances can greatly increase the risk of overdose. Use of a potent drug in a lozenge dosage form provides a method for administering a drug such as fentanyl to a cancer patient, and allowing the patient or caregiver to administer as much of the drug as is needed. This method of titrating the dose according to the patient's needs reduces the risk of overdose.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed and desired to be secured by United States Letters Patent is:

1. A dissolvable drug-containing dosage-form for use in the transmucosal delivery of a drug to a patient, comprising:
a) a binding agent that is formed into a solid matrix dissolvable in the mouth of a patient;
b) a pharmacologically effective dose of a potent central nervous system affecting drug that can be absorbed through mucosal tissues of the mouth, pharynx, and esophagus, the drug dispersed throughout the solid matrix; and
c) a buffer dispersed throughout the solid matrix, the buffer capable of controlling the pH of the mouth, pharynx, and esophagus to optimize permeation of the drug in order to facilitate transmucosal absorption of the drug;
wherein when the solid matrix dissolves in the mouth of the patient, the pharmacologically effective dose of the drug is released for absorption through mucosal tissues of the mouth, pharynx, and esophagus of the patient.

2. The drug-containing dosage-form of claim 1, wherein the binding agent comprises a carbohydrate that is dissolvable in the mouth of the patient.

3. The drug-containing dosage-form of claim 2, wherein the carbohydrate is selected from the group consisting of a cellulose, a starch, a sugar, derivatives thereof, and mixtures thereof.

4. The drug-containing dosage-form of claim 1, wherein the binding agent comprises a fat that is dissolvable in the mouth of the patient.

5. The drug-containing dosage-form of claim 1, wherein the binding agent comprises a protein that is dissolvable in the mouth of the patient.

6. The drug-containing dosage-form of claim 5, wherein the protein comprises a gelatin.

7. The drug-containing dosage-form of claim 1, wherein the binding agent comprises a hydrocarbon that is dissolvable in the mouth of the patient.

8. The drug-containing dosage-form of claim 1, wherein the binding agent comprises a wax.

9. The drug-containing dosage-form of claim 1, wherein the binding agent comprises a hydrogel.

10. The drug-containing dosage-form of claim 1, wherein the solid matrix is a compressed powder.

11. The drug-containing dosage-form of claim 1, wherein the drug is microencapsulated.

12. The drug-containing dosage-form of claim 1, wherein the drug is contained within a sponge-like material that is biologically inert and capable of entrapping a drug and then releasing the drug over time.

13. The drug-containing dosage-form of claim 1, wherein the drug is dispersed substantially uniformly throughout the binding agent.

14. The drug-containing dosage-form of claim 1, wherein the drug is contained within the binding agent.

15. The drug-containing dosage-form of claim 1, wherein the drug adheres to the binding agent.

16. The drug-containing dosage-form of claim 1, wherein the drug is an analgesic.

17. The drug-containing dosage-form of claim 1, wherein the drug is an anesthetic.

18. The drug-containing dosage from of claim 1, wherein the drug is an anxiolytic.

19. The drug-containing dosage-form of claim 1, wherein the drug is a sedative.

20. The drug-containing dosage-form of claim 1, wherein the drug has opioid agonist effects on the patient.

21. The drug-containing dosage-form of claim 1, wherein the drug has opioid antagonist effects on the patient.

22. The drug-containing dosage-form of claim 1, wherein the drug is selected from the group consisting of fentanyl, sufentanil, lofentanil, carfentanil, alfentanil, or mixtures thereof.

23. The drug-containing dosage-form of claim 1, wherein the drug is selected from the group consisting of codeine, morphine, or mixtures thereof.

24. The drug-containing dosage-form of claim 1, wherein the drug is a benzodiazepine.

25. The drug-containing dosage-form of claim 1, wherein the drug is selected from the group consisting of midazolam, triazolam, oxazolam, diazepam, oxazepam, lorazepam, or mixtures thereof.

26. The drug-containing dosage-form of claim 1, wherein the drug is selected from the group consisting of phencyclidine, ketamine, propanidid, propofol, thiamylal, or mixtures thereof.

27. The drug-containing dosage-form of claim 1, wherein the drug is a barbiturate.

28. The drug-containing dosage-form of claim 1, wherein the drug is selected from the group consisting of thiopental, methohexital, pentobarbital, hexobarbital, and mixtures thereof.

29. The drug-containing dosage form of claim 1, wherein the drug is a cannobinoid.

30. The drug-containing form of claim 1, wherein the drug is selected from the group consisting of Δ⁹ THC, CP 55,940, WIN 55,212 and levonantradol, and mixtures thereof.

31. The drug-containing dosage-form of claim 1, wherein the drug is substantially ionizable and nonlipophilic.

32. The drug-containing dosage-form of claim 1, wherein the buffer comprises a citrate buffer system.

33. The drug-containing dosage-form of claim 1, wherein the buffer comprises a phosphate buffer system.

34. The drug-containing dosage-form of claim 1, wherein the solid matrix further comprises a lubricating agent.

35. The drug-containing dosage-form of claim 1, wherein the solid matrix further comprises a surfactant.

36. The drug-containing dosage-form of claim 1, wherein the solid matrix further comprises maltodextrin in order to aid in dissipating any unpleasant flavors of the drug in the solid matrix.

37. The drug-containing dosage-form of claim l, wherein the solid matrix further comprises a hydrophobic agent in order to slow the dissolution of the solid matrix in the mouth of the patient.

38. The drug-containing dosage-form of claim 1, wherein the solid matrix further comprises at least one flavoring.

39. The drug-containing dosage-form of claim 1, wherein the solid matrix further comprises at least one sweetener.

40. The drug-containing dosage-form of claim 1, further comprising a permeation enhancer dispersed throughout the solid matrix, the permeation enhancer capable of modifying the permeability of the drug in the mucosal tissue in order to facilitate transmucosal absorption of the drug.

41. A nondissolvabe drug-containing dosage form for use in transmucosal delivery of a drug to a patient, comprising:
a) a drug containment matrix that is nondissolvable in the mouth of the patient; and
b) a pharmacologically effective dose of a central nervous system affecting drug that can be absorbed through mucosal tissues of the mouth, pharynx, and esophagus, the drug being incorporated into the nondissolvable drug containment matrix which is configured to release the drug within the mouth of the patient for absorption through mucosal tissues of the mouth, pharynx, and esophagus.

42. The drug-containing dosage-form of claim 41, wherein the nondissolvable drug containment matrix includes a chamber defined by a permeable barrier having a pore size sufficiently large to permit passage of drug molecules there through under appropriate conditions.

43. The drug-containing dosage-form of claim 41, wherein the drug is micro encapsulated.

44. The drug-containing dosage-form of claim 41, wherein the drug is contained within a sponge-like matrix which entraps the drug and releases the drug within the mouth of the patient over time in response to pressure exerted on the sponge-like matrix by the mouth of the patient.

45. The drug-containing dosage-form of claim 41, further comprising a biocompatible material to adhere together a plurality of microencapsulated drug particles into a preselected shape.

46. The drug-containing dosage-form of claim 41, wherein the drug is selected from the group consisting of fentanyl, sufentanil, lofentanil, carfentanil, alfentanil, or mixtures thereof.

47. The drug-containing dosage-form of claim 41, wherein the drug is selected from the group consisting of codeine, morphine, or mixtures thereof.

48. The drug-containing dosage-form of claim 41, wherein the drug is a benzodiazepine.

49. The drug-containing dosage-form of claim 41, wherein the drug is selected from the group consisting of midazolam, triazolam, oxazolam, diazepam, oxazepam, lorazepam, or mixtures thereof.

50. The drug-containing dosage-form of claim 41, wherein the drug is selected from the group consisting of phencyclidine, ketamine, propanidid, propofol, thiamylal, or mixtures thereof.

51. The drug-containing dosage-form of claim 41, wherein the drug is a barbiturate.

52. The drug-containing dosage-form of claim 41, wherein the drug is selected from the group consisting of thiopental, methohexital, pentobarbital, hexobarbital, or mixtures thereof.

53. The drug containing dosage form of claim 41, wherein the drug is a cannabinoid.

54. The drug-containing form of claim 41, wherein the drug is selected from the group consisting of Δ⁹ THC, CP 55,940, WIN 55,212 and levonantradol, and mixtures thereof.

55. The drug-containing dosage-form of claim 41, wherein the drug is a lipophilic drug.

56. The drug-containing dosage-form of claim 41, wherein the drug is a nonlipophilic drug.

57. The drug-containing dosage-form of claim 41, further comprising a buffer held within the drug containment matrix, the buffer capable of modifying the salival pH when dissolved in saliva such that a majority of the drug remains unionized in order to facilitate transmucosal absorption of the drug.

58. The drug-containing dosage-form of claim 41, wherein the buffer comprises a citrate buffer system.

59. The drug-containing dosage-form of claim 41, wherein the buffer comprises a phosphate buffer system.

60. The drug-containing dosage-form of claim 41, further comprising a biocompatible adhesive for placing the dosage-form in a preselected position within the mouth of the patient.

61. The drug-containing dosage-form of claim 41, further comprising a permeation enhancer held within the drug containment matrix, the permeation enhancer capable of modifying the permeability of the mucosal tissues of the mouth, pharynx, and esophagus towards the drug in order to facilitate transmucosal absorption of the drug.

62. The drug-containing dosage-form of claim 41, wherein the permeation enhancer is selected from the group consisting of a bile salt, a bile salt analog, a bile salt derivative, and mixtures thereof.

63. A method of making a dissolvable drug-containing dosage-form for use in transmucosal delivery of a drug to a patient, the method comprising the steps of:
a) selecting a binding agent that is dissolvable in the mouth of a patient;
b) adding to the binding agent a pharmacologically effective dose of a potent central nervous system affecting drug, which can be absorbed through mucosal tissues of the mouth, pharynx, and esophagus of the patient;
c) adding a buffer to the binding agent, the buffer capable of controlling the pH of the mouth, pharynx, and esophagus to optimize permeation of the drug in order to facilitate transmucosal absorption of the drug;
d) mixing the binding agent, drug, and buffer into a moldable mixture in which the drug and enhancer are dispersed throughout the binding agent; and
e) forming a solid matrix from the moldable mixture that is dissolvable in the mouth of the patient such that the drug is released for absorption through mucosal tissues of the mouth, pharynx, and esophagus of the patient.

64. A dissolvable drug-containing dosage form for use in transmucosal delivery of a drug to a patient, comprising:
a) a binding agent that is formed into a solid matrix is dissolvable in the mouth of the patient;
b) a potent dose of a central nervous system effecting drug that can be absorbed through mucosal tissues of the mouth, pharynx and esophagus, the drug disbursed throughout the solid matrix; and
c) a buffer disbursed throughout the solid matrix, the buffer capable of controlling the pH of the mouth, pharynx and esophagus to optimized permeation of the drug in order to facilitate transmucosal absorption of the drug; wherein when the solid matrix dissolves in the mouth of the patient, the potent dose of the drug is controllably released for absorption through mucosal tissues of the mouth, pharynx and esophagus of the patient.

65. A drug-containing dosage form of claim 64, wherein the potent drug when compared to the weight of the solid matrix has a relatively low dose to matrix weight ratio.

66. The drug-containing dosage form of claim 64, wherein the potent drug accounts for less than about 5% of the dosage form weight.

67. The drug-containing dosage form of claim 64, wherein the potent drug accounts for less than about 3% of the dosage form weight.

68. The drug containing dosage form of claim 64, wherein the potent drug accounts for less than about 1% of the dosage form weight.

69. The drug-containing dosage form of claim 64, herein the potent drug accounts for less than about 0.5% of the dosage form weight.

70. The drug containing dosage form of claim 64, wherein the potent drug accounts for less than about 0.1% of the dosage form weight.

71. The drug containing dosage form of claim 64, wherein the potent drug accounts for less than about 0.05% of the dosage form weight.

72. The drug containing dosage form of claim 64, wherein the potent drug accounts for less than about 0.01% of the dosage form weight.

73. The drug containing dosage form of claim 64, wherein the potent drug is capable of being released slowly, over an extended period of time, to control absorption through mucosal tissue.

74. A dissolvable drug-containing dosage-form for use in the transmucosal delivery of a drug to a patient, comprising:
a) a binding agent that is formed into a solid carbohydrate matrix dissolvable in the mouth of a patient,
b) a pharmacologically effective dose of a potent central nervous system affecting drug that can be absorbed through mucosal tissues of the mouth, pharynx, and esophagus, the drug dispersed throughout the solid matrix; and
c) a buffer dispersed throughout the solid matrix, the buffer capable of controlling the pH of the mouth, pharynx, and esophagus to optimize permeation of the drug in order to facilitate transmucosal absorption of the drug;
wherein when the solid matrix dissolves in the mouth of the patient, the pharmacologically effective dose of the drug is released for absorption through mucosal tissues of the mouth, pharynx, and esophagus.

75. The dissolvable drug-containing dosage-form of claim 74 wherein the carbohydrate binding agent is selected from the group of cellulose derivatives, water-dispersible starch derivatives, and compressible sugars.

76. A dissolvable drug-containing dosage-form for use in the transmucosal delivery of a drug to a patient, comprising:
a) a binding agent that is formed into a solid carbohydrate matrix dissolvable in the mouth of a patient, said carbohydrate being selected from the group of microcrystalline cellulose and carboxymethyl cellulose,
b) a pharmacologically effective dose of a potent central nervous system affecting drug that can be absorbed through mucosal tissues of the mouth, pharynx, and esophagus, the drug dispersed throughout the solid matrix; and
c) a buffer dispersed throughout the solid matrix, the buffer capable of controlling the pH of the mouth, pharynx, and esophagus to optimize permeation of the drug in order to facilitate transmucosal absorption of the drug;
wherein when the solid matrix dissolves in the mouth of the patient, the pharmacologically effective dose of the drug is released for absorption through mucosal tissues of the mouth, pharynx, and esophagus.
